(19) United States
(12) Patent Application Publication
(10) Pub. No.: US 2009/0028947 A1
(43) Pub. Date: Jan. 29, 2009

(54) USING OF NANOSILVER IN POULTRY, LIVESTOCK AND AQUATICS INDUSTRY

(76) Inventor: JAFAR RAHMAN NIA, Tehran (IR)

Correspondence Address: BARRY CHOOBIN, 193 SUITE #18, TALEGHANI, BAHARE SHOMALI, TEHRAN, 1563714311, IR

(21) Appl. No.: 11/781,281

(22) Filed: Jul. 23, 2007

(51) Int. Cl. A61K 9/14; A61P 31/00

(52) U.S. Cl. 424/489

(57) ABSTRACT

Nanosilver material is disclosed. Said material is nano silver based disinfectant and disease preventer for poultry, livestock and aquatics. The material contains silver nano particle and pure water and can be used as a surface disinfectant, water disinfectant and therapentic material for poultry, livestock and aquatic disease caused by bacteria's viruses, fungi and other monocellular microorganism.

### STATEMENT REGARDING SPONSORED RESEARCH OR DEVELOPMENT

This invention was partially sponsored by Iranian National Science Foundation.

### FIELD OF THE INVENTION

The present invention relates to nanosilver based disinfectant and disease preventer coating silver nano particles and pure water.

### BACKGROUND OF THE INVENTION

Nanotechnology is a rapidly growing science of producing and utilizing nano-sized particles that measure in nanometers (1 nm=1 billionth of a meter). One nonmaterial that is having an early impact in healthcare products is nano-silver. Silver has been used for the treatment of medical ailments for over 100 years due to its natural antibacterial and antifungal properties. The nano-silver particles typically measure 25 nm. They have extremely large relative surface area, increasing their contact with bacteria or fungi, and vastly improving its bactericidal and fungicidal effectiveness.

The nano-silver when in contact with bacteria and fungus will adversely affect cellular metabolism and inhibit cell growth. The nano-silver suppresses respiration, basal metabolism of electron transfer system, and transport of substrate in the microbial cell membrane. The nano-silver inhibits multiplication and growth of those bacteria and fungi which cause infection, odor, itchiness and sores.

Nano-silver can be applied to a range of other healthcare products such as dressings for burns, scald, skin donor and recipient sites; plasters for surgical and trauma wounds; aqueous gel for spots, acne and cavity wounds; and female hygiene products
panty liners, sanitary towels and pants.
Nano-silver is:
   Highly efficacious
   Fast acting
   Non Poisonous
   Non stimulating
   Non allergic
   Tolerance Free
   Hydrophilic

Therefore, it would be advantageous to apply an active based material to poultry, livestock, and aquatic fields to disinfect and prevent disease.

### SUMMARY OF THE INVENTION

The present invention relates to a nano silver based disinfectant and disease preventer for use in poultry, livestock and aquatic industries.

This nano silver based active material contains 4000 ppm nano silver as silver nano particles and size distribution of said nano particles are 1 to 10 nm.

The nanosilver based active material has antibacterial and antifungal and antiviral effects. Usendo said nanosilver based active material; we can fight all bacteria fungi, viruses and other monocellular harmful microorganisms in filed of poultry, livestock and aquatics.

Furthermore, we can decrease or eliminate the use of drugs, especially antibiotics in said fields. In addition, we can reach a better weight gaining, decrease of fat in meat; decrease of cholesterol and increase of protein content of meat.

### DETAILED DESCRIPTION OF THE INVENTION

This invention relates to a nanosilver based, disinfectant and disease preventer material. This material contains following component.

Each ml. contains:

4 mg of silver nanoparticles, q.s to 1 ml of purified water and citric acid with the adjusting PH between 1-4.

The active material is silver in Nan-ionic form and in 1-10 nm size. Silver is completely stable carrier in form of suspension.

Its stability has been tested in accelerated and long terms studies. Silver is known antibacterial agent. Its nanoparticles can kill all kinds of bacteria in very low concentration (about 5 ppm). It is also an antiviral and can fight against various kinds of viruses.

In a low aquatic environment said active material can be used by spraying on the surface.

Silver acts as a photo catalyst. In the presence of air oxygen, it can produce free radicals, and these free radicals can effect the micro organisms and kill them.

In the high aquatic environment like water or in poultry, livestock and aquatic body, without air oxygen, silver nanoparticles penetrate into bacteria.

In this case, silver can affect the di-sulficdic bands, between protein complexes. Thus the 3-d form of protein changes and it can not perform as an enzyme. In case this happens in bacteria cell wall, the selective permeability of cell wall changes, therefore, bacteria swallows and will destroy. If this process occurs in energy cycle enzymes, then, the production of ATP stops and the lack of energy will kill microorganisms.

On viruses, the glycoprotein knobs of viral envelop, that are virus antigenic receptors, has a di-sulfic band. This band could be attacked by silver nanoparticles and can destroy the virus and prevents its attachment with host cell. With this mechanism of actions, silver nanoparticles can be used instead of all antibacterial agents and in preventive form; it can be even used as a vaccine.

In the absence of pathogenic microorganisms and their stress on immune system and other systems of poultry, livestock, and aquatics, the produced energy can be used to make more tissues and especially muscular tissues. This can improve weight gaining in poultry, livestock and aquatics.

It has been seen and experimented that use of silver nanoparticles probably can reduce the lipid and cholesterol contents of meat and can increase the protein contents of meat.

### EXAMPLES

### Example 1

Using of Nanosilver in Poultry Industry for Water Sanitation:

In an experiment, we mixed 3 liters of colloidal nanosilver with 4000 Liters of poultry drinking water. Microbiological tests showed that no microorganism exists in the water samples.

### Example 2

Using of Nanosilver in Aquatics Industry for Water Sanitation:

In an experiment, we mixed 2 Liters of nanosilver colloid with 4000 Liters of aquatics pool water. Microbiological tests showed that no microorganism exists in the water sample.

### Example 3

Using of Nanosilver in Sterilization of Surfaces in Poultry Industry:

We mixed 5 Liters of nanosilver colloid with 4000 Liters of water and applied this mixture to walls and sealing of aviary. Samples collected from these surfaces and microbiological tests done. These tests showed that no microorganism exists in these surfaces after applying the mixture.

### Example 4

Using of Nanosilver in Livestock Industry for Water Sanitation:

In an experiment, we mixed 3 liters of colloidal nanosilver with 4000 Liters of livestock drinking water. Microbiological tests showed that no microorganism exists in the water samples.

### Example 5

Using of Nanosilver in Sterilization of Surfaces in Livestock Industry:

We mixed 5 Liters of nanosilver colloid with 4000 Liters of water and applied this mixture to walls and sealing of livestock stable. Samples collected from these surfaces and microbiological tests done. These tests showed that no microorganism exists in these surfaces after applying the mixture.

### Example 6

Using of Nanosilver in Poultry Industry for Feed Sanitation: We mixed 10 ml of nanosilver colloid with 100 Kg of feed. Microbiological tests done, after sampling. These tests showed that no pathogenic microorganism exists in samples.

### Example 7

Using of Nanosilver for Prevention of Diseases in Poultry Industry:

We used nanosilver colloid in aviaries, according to example 1, 2 and 6. We used nanosilver colloid in feed (10 ml of nanosilver colloid in 100 Kg of feed), drinking water (3 Liters of nanosilver colloid in 4000 Liters of water) and surfaces (5 Liters of nanosilver colloid in 4000 Liters of water). In a full growth period (50 days), poultry grew without using of any drug such as antibiotics and no vaccine and without any problem with bacterial, fungal or viral diseases.

The invention will be apparent to those skilled in the art from a consideration of the specification or practice of the invention disclosed herein. It is intended that the specification and examples be considered as exemplary only, with the true scope and spirit of the invention being indicated by the following claims.

What is claimed:

1. A nanosilver based active material for disinfection and disease prevention for poultry, livestock and aquatics, said active martial consisting of; A) nanosilver particles, wherein distribution of said nanosilver particles is between 1 to 10 nm;
B) purified water; and
C) citric acid.

2. The nanosilver based active material as claimed in claim 1, wherein said nanosilver based active material is sprayed to further disinfect surfaces.

3. The nanosilver based active material as claimed in claim 1, where in said active material further sanitizes water, food, and wherein said water and food are used in poultry, livestock and aquatic industry.

4. The nanosilver based active material as claimed in claim 1, wherein said active material prevents disease caused by bacteria viruses, fungi, and mono cellular microorganisms.

5. The nanosilver based active material as claimed in claim 1, wherein said active material eliminates needs of using drugs in poultry, livestock and aquatic industries.

6. The nanosilver based active material as claimed in claim 1, wherein said active material reduces lipids and cholesterol in poultry meat.

7. The nanosilver based active material as claimed in claim 1, wherein said active material increases protein in poultry meat.

8. The nanosilver based active material as claimed in claim 1, wherein said active material enhances weight gaining in poultry, livestock and aquatics.
